(19) United States
(12) Patent Application Publication
(10) Pub. No.: US 2022/0010262 A1
(43) Pub. Date: Jan. 13, 2022

(54) Method For Dissociation Of Cells

(71) Applicant: Grain Processing Corporation, Muscaline, IA (US)

(72) Inventor: Sarjit Johal, Iowa City, IA (US)

(21) Appl. No.: 17/369,390

(22) Filed: Jul. 7, 2021

Related U.S. Application Data
(60) Provisional application No. 63/050,964, filed on Jul. 13, 2020.

(51) Int. Cl. C12N 1/02; C08B 37/00

(52) U.S. Cl. CPC C12N 1/02; C12N 2521/00; C12N 2523/00; C08B 37/0003

(57) ABSTRACT

Disclosed is a method for the dissociation of cells. Cells are processed under varying conditions of pH, temperature, and shear to thereby produce different cell products. In one form, the cells are jet cooked at a lower temperature and/or pressure to provide products that are relatively delicate. The remaining cell components may then be subsequently jet cooked under higher temperature and/or shear conditions to provide products that are relatively more robust. Generally, the cells become dissociated, whereby at least one separate cell wall component is substantially separate from the dissociated cell walls.

### CROSS-REFERENCE TO RELATED APPLICATION

This application claims benefit of U.S. Provisional Application No. 63/050,964, filed Jul. 13, 2020, which is hereby incorporated by reference in its entirety.

### FIELD

This application relates to dissociation of cells to obtain nutrients and other useful products therefrom, including using processing conditions to obtain specific cellular components.

### BACKGROUND

Yeast and yeast metabolites are widely used in an array of food and feed products. Baker's and brewer's yeast, for example, are excellent sources of nutrients and flavoring agents. Nutrients that are obtainable from cells include insoluble and soluble cell wall polysaccharides, oligosaccharides, glucans, proteins, peptides, nucleotides, and the like. Cells, in particular cell walls, are also thought to absorb pathogens and consequently to provide a measure of prophylaxis against infection.

Cell disruption breaks the cells and improves accessibility to the intracellular components for extraction. It can be important for the purification of intracellular and cell wall biomolecules. Lysed cells and cell fractions are thought to contain many nutritive components in a form that is bio-available to the consuming animal. Live yeast cells are thought to aid in digestion in ways not fully understood at present. Whole dead cells, on the other hand, are not thought to be of particular nutritive benefit, except possibly in ruminant animals. The digestive tract of monogastric animals is essentially unable to rupture the cell wall, and thus the majority of the dead cells pass through the digestive tract and are typically excreted whole, without releasing nutrients to the animal.

A number of methods are known for rupturing yeast cells, including, but not limited to, mechanical, hydrolytic and autolytic methods. Mechanical methods typically are employed in small-scale laboratory applications. Conventional mechanical disruption includes presses, such as the French press, homogenizers, sonic disruptors, and so forth. In a laboratory French press, for example, pressures as high 20,000 psi and high shear conditions are produced by passing the cells through a small orifice. Other devices subject the cell to different stresses but provide the same result, that is, rupture of the cell wall. For instance, another known apparatus, the bead beater, contains ceramic or glass pellets that are used to crush, shear, and fracture cells. Hydrolytic procedures employ enzymes, acid, or alkali to rupture the cell walls. Cell autolysis is a well-known process wherein the yeast cell is subjected to digestion by its own enzymes.

Autolysis is the most recognized and widely used cell disruption practice. Autolysis does not dissociate or crack the yeast cell wall. Rather the methodology punctures the cell wall which results in the discharge of soluble cytoplasm into the media. However, the unrestrained enzymatic reactions degrade biomolecules which results in a low molecular weight composition termed yeast extract. Hydrolytic yeast extracts are concentrates of the soluble materials after digestion (lysis) by proteases, nucleases and other hydrolytic enzymes in the cell. The broad destruction of intracellular structure and components does facilitate solubilization and discharge of the degraded cytoplasmic constituents. Chaotropic agents such as sodium chloride are also used to enhance release of intracellular components into the media.

The most traditional form of yeast protein is yeast extract. These are primarily used by the food industry as flavorings. The manner of autolysis affects the flavor profile of the extract. The destruction of the native structure also substantially alters the functional and nutrition composition of released materials and make it highly unlikely that this converted heterogeneous cell mass can be fractionated or purified. Ultimately, this limits their utility, potential and value. For instance, yeast extract/proteins are not competitive with soy, pea, wheat or other proteins.

The cell wall, the other major product of autolysis, is typically not processed and contributes little value as a by-product. To date, the perforated cell walls are generally viewed as a low value ruminant feed or pet palatant.

Other cell disruption methods can be divided into two groups: (i) mechanical and (ii) non-mechanical. Disruption of the cell wall in a non-specific manner is achieved by mechanical means including physicochemical forces such as shear, high pressure, heat and combinations thereof. Non-mechanical methods such as chemical and enzymatic procedures while judged to be more benign often only perforate rather than dissociate cells. The non-mechanical approaches rely upon selective interactions between an enzyme or chemical agent and a specific cell marker or wall component. The reaction at the cell barrier allows soluble biomolecules to seep out of the cell as in autolysis.

While there are many laboratory scale rupture methods in the literature, few can be scaled cost effectively for industrial applications. For instance, most enzymatic and chemical treatments are slow and costly, while handling and disposal of process additives may be difficult. Mechanical methods resolve some of the challenges posed by non-mechanical methods but have their own issues. For instance, mills, presses and homogenizers can reduce unit operation steps compared to enzymatic and chemical methods but they do little to reduce costs or substantially improve productivity. Further, as with the non-mechanical methods, the value of the by-products is negligible as they are often degraded and are not amenable to fractionation and purification. Nonetheless, mechanically processed by-products can have limited use in select applications such as flavorings, fillers, and generic sources of nutrients.

The enzymatic methods are also inherently limited in their capacity to obtain the value of the constituents as each methodology is selective and targeted. Further, while enzymatic processes can be used to manufacture specific cytoplasmic or wall entities, they have limited capacity to dissociate the cell wall or recover structurally intact biomolecules. Conversely the commercially scalable mechanical processes, while potentially capable of dissociating the cell wall, typically destroy the labile cytoplasmic elements in the process. Sequentially running both processes as is now the case is expensive and inefficient except for the manufacture of high value active ingredients.

It is desirable if the two major cell fractions, the intracellular constituents and cell wall components, could be separated and recovered in an integrated process. Thermal processes for the lysis and dissociation of biological materials such as microbial, fungal and plant biomass have been extensively studied. Relative to other technologies, thermal processes offer many commercial opportunities including equipment availability, low cost, extensive knowledge and experience. Autoclaves, steam explosion, heat exchangers and high temperature systems are all examples of thermal methods.

U.S. Pat. No. 7,425,439 utilizes jet cooking for the purpose of dissociating cells and demonstrated that jet cooking offered significant advantages and efficiencies including but not limited to capital, material costs, and operational flexibility. It also revealed finished product qualities not available with other thermal such as autoclaving. The technology ruptured the cell and dissociated the cell wall quickly and in a manner that released the soluble cytoplasmic components as well as the major cell wall constituents including glucans, mannan-oligosaccharides, minor components and metabolites. While a significant improvement over available cell dissociation technologies, the technology did little to enable or simplify separation of the cell's cytoplasmic or wall fractions. Instead the reference taught that all of the constituents be dried without taking into account structural and chemical differences that define functional properties such as solubility, maximum temperature tolerance and nutritional properties.

It would be commercially beneficial to separate the cell elements into usable, and potentially more valuable discrete components. It would be particularly advantageous if the more labile fraction such as the yeast extract or soluble intracellular portion could be recovered early in the cell dissociation process prior to treatment of the wall as this fraction is resistant to disruption and requires harsher conditions. Further, it would be helpful if different cell types could be co-processed in a single system to yield a combination of components from multiple cell types.

### SUMMARY

In one form, a cell dissociation technology has now been developed that may resolve these challenges. The technology disclosed may also enhance the recovery of complex, heterogeneous biomolecules as well as labile, low molecular weight metabolites without endozymes or exozymes in a simplified manner. In one form, the methods also allow separation of the major fractions devoid of a yeast extract digest. If a digest is required, it can be produced by the addition of highly selective enzymes to a defined intra or wall fraction.

Microorganisms can be separated into multiple fractions based on one or more physicochemical properties. For example, the yeast cell can be separated into soluble and insoluble constituents. Each of these can be separated further. The soluble fraction for instance can be divided into low and high molecular weight entities at low temperature conditions. The soluble fraction can be dried or separated further using mild conditions that do not degrade the constituents. The insoluble fraction can also be further divided by using greater heat and shear. The insoluble fraction which contains cell wall fragments and robust complexes will require more energy and heat in order to effect dissociation. Further, the insoluble portion may need to undergo one or more recovery steps as the readily dissociated constituents in the initial composition might be irreversibly damaged during the next harsher step or treatment stage. This multistep cycle, if done in a continuous stepwise manner, could yield enhanced separation with less denaturation resulting in greater purity within the fractions. In yeast and related unicellular organisms for instance the terminal stage might result in higher concentration of the major cell component, glucan and mannan oligosaccharides. These polysaccharides could be used ‘as is’ or further separated. Whether purified or used ‘as is’ these cell wall constituents could yield better functionality.

It has now been found that yeasts, fungi, bacteria, and other cells (including eukaryotic cells) may be processed to recover soluble or insoluble cell components such as proteins, saccharides, peptides, lipids, glucans, and the like. Generally, the cells are processed by a shearing force in the presence of heat. However, at least in a first stage, the shear and heat used is lowered relative to prior methods so that relatively sensitive and/or delicate materials may be obtained without substantially degrading the materials, as had occurred in prior methods.

In accordance with one form, a method for dissociating cells is provided. In one form, conditions of pH, shear, and temperature suitable for extraction of one or more cell components are selected. The method is intended to at least partially remove and separate relatively sensitive and/or delicate materials in a first stage. Subsequent stages may be used to remove and separate other more robust materials from the cells.

The method preferably comprises jet cooking the cells. In the most highly preferred embodiments of the invention, the cells are jet cooked to form an intermediate product, and the intermediate product is subsequently jet cooked to form the mixture of cytoplasm and cells. The mixture thus formed may be spray dried or otherwise treated, such as by substantially separating the cell walls from the cytoplasm. An animal feed may be prepared from the mixture or spray dried mixture thus formed.

### BRIEF DESCRIPTION OF THE DRAWINGS

FIG. 1 is a process flow diagram showing removal of a soluble co-product in an initial processing stage; and

FIG. 2 is a process flow diagram showing a single stage dissociation and fractionation of soluble and insoluble microbial and biomass elements.

### DETAILED DESCRIPTION

The present disclosure relates to concepts to permealize and recover a substantially native cell cytoplasm enriched media as well as a dissociated cell wall fraction. In practice the soluble polymeric cytoplasmic constituents are released and separated from the insoluble cell wall material without further treatment and dried. The remaining insoluble cell biomass may then be slurried and immediately subjected to a thermochemical treatment that fluidizes and dissociates the wall components.

According to one form, a multi-stage process can be used to recover one or more products. In a first stage, the cell is weakened and destabilizes the entire cell wall. In this form, unlike autolysis where the microbes own hydrolytic enzymes destroy the cell internally and in the process puncture the wall, the present disclosure uses a simple, rapid means to weaken and breach the wall using mild conditions from which cell rupture proceeds. In other words, in one form, the present disclosure seeks to create an external tear in the wall which is sufficient to release the cytoplasmic fraction as well as prepare the insoluble wall fraction for dissociation.

In the next stage, harsher conditions of temperature, pH, shear and other forces dissociate the cell wall and discharge the tightly bound constituents. In stage 1, the aim is to use methodologies that limit degradation so that the primary structure of the biomolecules will not be degraded. Following recovery of the soluble fraction, the insoluble wall and associated components are processed to recover embedded polysaccharides including, but not limited, to glucans, mannan, proteins, lipids and lower molecular weight biomolecules. In one form, this can best be accomplished by jet cooking. In this manner the labile cytoplasmic fraction can be recovered as well as the cell wall dissociated in a quick, efficient and economical manner.

Unlike other teachings which rely on multiple, lengthy processes, the concepts discussed herein can be used for cell dissociation by reducing both the number of steps as well as using efficient procedures that are directed at the production of structurally intact, distinct populations of biomolecules. According to one form, the concepts discussed herein can be used with native yeast and microbial proteins, nucleic acids, polysaccharides and lipids. In addition to being more efficient, the separated intact biomolecules represent new compositions for further application development. In some forms, the processes used herein can provide for a continuous cell dissociation and separation system that is scalable using commercially available equipment and produce multiple product streams without the use of enzymes or harsh chemicals such as alkalis or acids.

Cell Types.

The methods, processes, and systems described herein may be used with a variety of different types of cells to recover different products therefrom. The disclosure herein may be applicable to any prokaryotic or eukaryotic cells, in particular microbial cells, and especially to yeasts. Other cells suitable for dissociation in connection with the present inventive method include bacteria, fungi, algae, seaweed, and plant cells, mushroom and spores. More generally, any cell that can be “harvested” to provide nutrients or other chemically useful materials can be used in conjunction with the invention. If yeast is used, the preferred strains include Saccharomyces, Torula, Pichia, Kluveromyces, Schizosaccharomyes and others. Many of these are used and sold commercially. Saccharomyces cerevisiae and Torulopsis utilis being the best known. The commonly recognized types of S. cerevisiae include Baker's, Brewer's, Nutritional as well as Distillers and Wine yeast. It should be appreciated that other forms of yeast may also be used alone or in combination with other yeasts or other cell types.

The cells may be alive or dead, or mixtures of live and dead cells may be employed. The yeast cells may be used as supplied from a commercial distilling operation, or may be washed prior to use in conjunction with the invention to remove bittering agents, fermentation insolubles, and the like. It is contemplated that the yeast may include fiber, carbohydrate, or other material from a commercial ethanol distilling operation, and in some embodiments of the invention the yeast source may comprise stillage. Preferred yeast sources are liquid, dried, or compressed yeast.

In addition to yeast, other microorganisms that can be ruptured and dissociated using the inventive process include bacteria, photosynthetic cyanobacteria, unicellular algae and related plant like autotrophs that contain chlorophyll such as Euglena, green algae and diatoms. Diatoms are unicellular algae that have siliceous cell walls and comprise a very large number of species.

Elements of a microorganism can contribute both macro and micro nutrients as well as distinct biomolecules possessing functional properties that can regulate biochemical pathways, aid in digestion, and well-being. In the yeast cell wall for instance the glucan component can rouse an immune response and the mannan oligosaccharide can bind and remove select pathogens in the gut. In contrast most bacterial biomass which does not possess these polysaccharides has a protein content that ranges from 50-80%. Often this biomass also has a superior amino acid content. Alone these two materials offer advantages to an animal however, if co-processed the finished composition would contain both properties at a substantially low cost.

It should also be appreciated that multiple different cells and/or microorganisms can be processed at the same time. In this regard, a single processing operation can be used on a combination of multiple different types of cells. It is believed that such a combined processing operation may yield improved resulting products than if the different types of cells were processed separately and then recombined. As discussed herein, the various different cells and microorganisms can be fractionated and/or processed using a gradient for the conditions. In other words, lower shear, lower temperature, and the like can be used in an initial processing step to obtain delicate materials and/or materials from cells or organisms that are delicate. Examples of low molecular metabolites susceptible to denature at moderate to high temperatures include but are not limited vitamins, specifically water soluble vitamins such as Vitamin C and the Vitamin B complex: thiamin (B1), riboflavin (B2), niacin (B3), pantothenic acid (B5), Vitamin B6, biotin (B7), folic acid (B9), Vitamin B12; select nucleic acid, complex bio assemblages, organelles such chloroplasts, mitochondria. This rupture or dissociation of these bodies is in itself perhaps not critical, but the dissociation contaminates the extract and thereby substantially complicates fractionation and purification of other biomolecules. Rupturing a chloroplast will for instance will release colored photosynthetic pigments into the supernatant which are difficult to remove and may contaminate the finished product.

Specific biomolecules may be sought as products from the cells. Such biomolecules may include, but are not limited to, proteins, nucleic acids, polysaccharides, fatty acids and low molecular weight and bioactive metabolites such as peptides.

Process Stages and Conditions

As noted above, the process can be carried out in one or more steps, depending on the cells, the types of materials that are being recovered, and the like. For example, if the only material being sought is cytoplasm and the soluble constituents, the process may be carried out in a single step or stage. In other forms, where multiple distinct materials are sought, such as cytoplasm separated from cell walls, multiple steps or stages can be carried out. Multiple steps or stages may be necessary where the desired products have different properties and/or are otherwise susceptible to different processing conditions.

Any suitable apparatus may be employed in connection with the methods described herein. In accordance with one embodiment, a jet cooking apparatus is employed. A jet cooking apparatus resembles a jet pump that is employed to move liquids and slurries. In the jet cooking process, saturated steam is injected through a nozzle into the center of a venturi mix combining tube. The slurry is then pulled into the annular gap formed by the steam nozzle and the venturi opening. The slurry is heated as it accelerates within the mixing tube. While passing through the mixing tube, the cells are subjected to extremely turbulent conditions which can cause partial hydrolysis of the cell walls, depending on the processing conditions.

It is contemplated in preferred embodiments of the invention that multiple passes through a jet cooking apparatus, preferably between 2 to 5 passes, and more preferably 2 to 3 passes, will be employed. If it is desired to completely liquefy the cells, i.e., to disassociate the cells to an extent such that the cell walls are substantially completely dissociated with no intact ghosts remaining, a higher number of passes, such as 3 to 7, may be employed. The precise number of passes required to achieve complete dissociation and the number of passes required to achieve a mixture of cytoplasm and ghosts will depend upon the specific apparatus employed and on the other operating conditions. It should be appreciated that when multiple passes are used having generally similar processing conditions, those multiple passes may be considered as a single stage.

In one form, the process may include two stages. The first stage is executed under relatively mild conditions of temperature, pH, shear, with optional use of select chaotropic agents and/or short, low dose exposure to with enzymes.

In a first stage, typical temperature conditions may generally be kept significantly lower than the temperatures used in typical jet cooking operations, such as found in U.S. Pat. No. 7,425,439. Instead, the temperature may be from about 140° F. to about 250° F. According to one form, the temperature range may be from about 86° F. to about 167° F. In other forms, the temperature may range from about 170° F. to about 225° F. In later stages, the temperature may be increased to extract other cell components that are not as sensitive.

As noted above, the temperature range may be adjusted depending on a number of variables including, but not limited to, the desired product, the amount of shear, the pH, if chaotropic agents are used, and the like. In one form, when soluble, low molecular weight metabolites is desired as the product, the temperature should generally be kept below about 150° F. so as to not denature or otherwise damage the desired product. Generally, the products sought from a single step process or from the first step of a multi-step process are more susceptible to damage from heat such that the temperature is kept relatively low compared to prior jet cooking operations.

In one form, the use of temperature may by graduated. More specifically, the temperature may be increased at different times to obtain different products throughout the one or more steps of the process. A first step may have a low temperature range to obtain more delicate products whereas a second step may use a higher temperature range to obtain less delicate products.

The temperature may be controlled in a number of manners. For instance, in one form, heat may be added by using a jet cooking or modified jet cooking process to provide the desired temperature range. Other processes for adding heat and/or otherwise controlling the temperature may be used including, but not limited to, various types of heat exchangers, direct steam injection systems, in-line sanitary heaters and related systems.

The second or later stages may use much higher temperatures to generally break down other materials that are the least delicate, such as breaking down cell walls. For instance, much higher temperatures may be used, similar to temperatures used in traditional jet cooking processes.

Similarly, pressure and/or shear may be kept relatively lower during a first stage and then increased in later stages. For example, in one form, the pressure in a jet cooking operation in a first stage is between about 10 psi and 50 psi. In later stages, the pressure may be increased such as between about 50 psi and 80 psi.

Generally, the cells may be subjected to a pressure of between 35 to 105 psig at the conditions of temperature, pH, and shear heretofore discussed. The cells are preferably subjected to such pressure for a time ranging from 10 to 150 seconds. Once again, this parameter will be expected to vary with the other operating parameters.

The second and/or subsequent stages may provide a more complete dissolution of the cells. In one form, the walls of cells are dissociated to yield cell wall components. The dissociation contemplates a wide range of dissociation of the cell walls, and the extent of dissociation may be selected by one of skill in the art. For instance, the cells as received may contain impurities or non-native components that are bound via electrostatic forces (or even covalent bonds) to the cell walls. The dissociation in some embodiments of the invention contemplates removal of these impurities or non-native components. In preferred embodiments of the invention, the cell walls are partially disintegrated, such that some native cell wall components have been liberated from the molecular structure of the cell walls, but that the cell wall ghosts are still discernable as discrete entities under microscopic examination. It is thus contemplated that the ghosts may not be complete cell walls, inasmuch as some of the original components of the cell wall may have become dissociated from the remaining components of the cell wall. Any portion of native cell wall components may be so liberated, such as 10%, 20%, 30%, 40%, 50%, 60%, 70%, 80%, or 90%, whereby in such embodiments, the cell wall ghosts are still discernable. In less preferred embodiments of the invention, the dissociation is completed to an extent such that the cell walls are substantially completely or fully disintegrated, such that the cell walls are not visible as discrete entities under microscopic examination.

In carrying out the method, the pH of the slurry of cells is adjusted to any suitable pH, preferably a pH between 8.0 and 12.0, more preferably 9.0 to 11.0, and most preferably 9.5 to 10.0, using an alkali agent, most preferably a food-grade alkali such as sodium hydroxide, calcium hydroxide, or potassium hydroxide. The methods are not limited to processing under alkaline conditions. In some embodiments, strongly acidic conditions, preferably pH 0.5 to 3, and more preferably pH 1 to 2, may be employed. The preferred acidifying agent is a food-grade acid, such as hydrochloric, phosphoric, sulfuric, or mixtures thereof. Because it is believed in most instances that an acid pH is far more aggressive than the relatively mild alkaline conditions that may be employed for alkali hydrolysis of the yeast, alkaline conditions are preferred in connection with the present invention.

pH can facilitate the dissociation process in several ways. First, the pH of the liquid contacting the cell surface can fundamentally alter the wall and membrane chemistry. In response to acids, changes in wall and membrane permeability, anion extrusion as well as other alterations signal shifts in interaction between major cell components. Similarly, responses of cells to alkaline pH are known.

Wide fluctuations in pH can also affect particular properties of biomolecules. For instance, the solubility of beta glucan, a major component of yeast cell walls, is greater at alkaline pHs than at acidic conditions. Subjecting cells walls to acid and alkaline stress can induce changes in wall properties. The preferred order of stressing the walls is first treat with acid, pH range 3.0 to 5.0, more preferably 3.5 to 4.5 then alkaline exposure at pH range of 8 to 12, more preferably 9 to 11.

Other additives and/or agents may also be used. These materials may include, but are not limited to salt(s) and related osmotic stressors, acids, bases, chemical denaturants such as detergents, urea, peroxides as well as controlled enzyme reactions. In one form, one or more of the additives, alone or in combination with other agents and/or additives may be sufficient to permeabilize the cell membrane or wall in the presence of agitation or shear. Examples of multicomponent compositions can consist of naturally occurring food grade surfactants, such as lecithin from egg yolk and various proteins from milk, polar lipids such as monoglycerides. Synthetic surfactants such as sorbitan esters and their ethoxylates are also used in food emulsions in combination with various salts.

The activity of the additives/agents can be fine-tuned by adjusting dosage, reaction time and conditions. Further the activity of these additives/agents can be fine-tuned to maximize release of large and small biomolecules. Different agents interact differently with cell constituents. Surfactants may, for example, exhibit greater reactivity towards cell wall components either by removing surface layers such as membranes or generally reacting to weaken cell walls. Salts such as sodium chloride, potassium chloride in addition to being osmatic stressors can enhance the solubility of select elements such as proteins by altering ionic strength of the media. It should be understood that agents and additives can be used not only in a single stage or a first stage of a multi-stage process, but also in later stages of multi-stage processes.

In one form, the first stage conditions can be adjusted to accommodate the composition and properties of the cell wall. One of more of the following physicochemical stressors including but not limited to temperature, pH, ionic strength, exposure time, shear, select enzymes directed to biomolecules of nominal interest such as lipases can be used to weaken and rupture thick cell walls. An assessment of cell wall structure and constituents can aid in fine tuning of disruption conditions. Prioritization and targeting of specific categories of biomolecules can further enhance this step as well as maximize the recovery and stabilization of targeted constitutes. This approach may not only aid in recovery, stabilization and potentially enhanced production of known material but higher purity.

The weakened porous cell wall is next subjected to one or more the following treatments including but not limited to high temperature, shear, pressurization, homogenization and/or some combination of reactions to maximize the release of constituents. The hollowed cell (or ghost) which is insoluble and cell content carried in the liquid is pumped to a liquid/solid separation system such as a drum filter, pressure filter, membrane filtration, and centrifugation or similar. The liquid and solids are separated. The liquid comprising the cell extract, is collected, concentrated and dried. Alternatively, the liquid can be fractionated prior to drying to recover different native biomolecules, metabolites and/or nutrients using known process technologies.

The solids stream, which consists primarily of insoluble cell wall and membrane elements, can be used as is or washed. If washed a dewatering step would be used prior to further processing. The insoluble liquid can be heated, pH adjusted, and jet cooked at a high temperature with high shear. The treated cell wall can also be homogenized both before and after jet cooking to maximize dissociation and aid in drying.

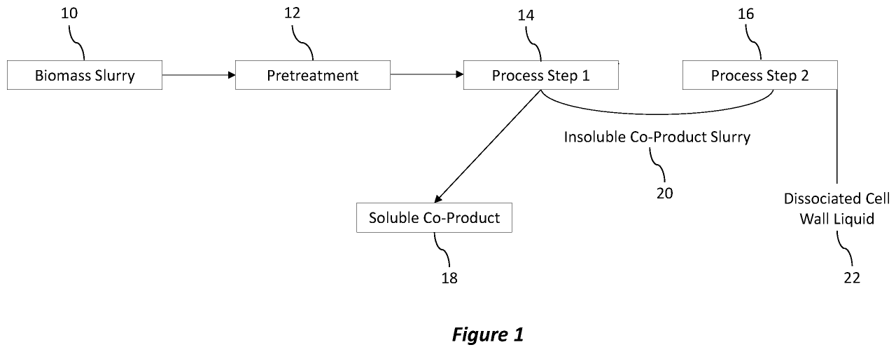

One form of a multi-step process is shown in FIG. 1. In this form, biomass slurry 10, which may include one or more different kinds of cells and/or microorganisms can be pretreated at step 12. Such pretreatment may include, but is not limited to, washing, pH adjusting, combination with additives/agents, such as surfactants, and the like. It should be appreciated that the pre-treatment 12 may also be optional.

The material is then subjected to a first processing step 14 to separate certain products, such as soluble materials. The first step 14 generally is less aggressive than later processing steps. However, the first step may include a variety of treatments and conditions. For example, a brief exposure to other non-denaturing cell disruption practices including but not limited to blenders, bead mills, ultrasonic devices or a combination thereof could be applied in the first step 14. Overall, the first step 14 is generally less aggressive than subsequent processing steps, such as by one or more lower temperatures and/or pressures, as outlined above.

The first step 14 may include repeated passes and/or batch processing, depending on the equipment and techniques used. For example, in one form, high pressure homogenization may involve single or multiple passes of a cell suspension through an adjustable, restricted orifice discharge valve. As the cells are forced at high pressure through the orifice they are subjected to liquid shear where operating pressure, cell concentration and temperature are influential upon disruption efficiency. Alternatively, other homogenizers recirculate the product back through a batch reactor for a specified blend time. Either approach is suitable to provide the necessary shear.

Like homogenization, mechanical cell disruption in a bead mill may also be used. Bead mills generally have characteristics including disruption efficiency in a single or multi-pass configuration, high throughput, as well as biomass loading. Bead mills are commercially available equipment ranging from laboratory to industrial scale. Cells are disrupted by shear forces generated by the radial acceleration of grinding elements (typically glass or zirconia beads) as well as by bead collisions. The rate of disruption is dependent on several operational parameters such as agitator speed, suspension throughput, bead size, bead loading and cell concentration.

In some embodiments, heat exchangers, thick film heating technologies and systems can be used to deactivate the cell to further reduce the possibility of the innate hydrolytic enzymes degrading either the soluble or insoluble fractions. A quick preheating step alone or in combination with a chemical denaturant may also weaken the cell wall and reduce the exposure to more extreme conditions or chemicals. This is particularly relevant to practices used in later stages where substantial disruption and cell wall dissociation preferred.

In some forms, a first stage may include jet cooking at relatively low temperature and/or shear while a second stage may include relatively higher temperatures and shear. This permits the more sensitive materials to be removed in the first stage while disrupting other components, such as cell walls in later stages. The temperatures and shear may be adjusted depending on the types of cells and the products desired. For example, in one form, the first stage 14 may include jet cooking at a temperature of less than 170° F., while a second stage 16 operates at a higher temperature, such as in a range of about 200° F. to about 300° F. In another form, more than two stages may be used. For example, a first stage may include jet cooking at a temperature less than 170° F., a second stage of jet cooking at a temperature range of about 170° F. to about 200° F., and a third stage of jet cooking at a temperature range of about 200° F. to about 250° F. In this gradient like approach, different materials may be extracted at different points throughout the multi-stage process. As shown in FIG. 1, a soluble co-product 18 may result from the first stage 14 while an insoluble co-product 20 is passed to the second stage 16. At the end of the second stage 16, dissociated cell wall material 22 may be produced as a result of the harsher conditions of the second stage.

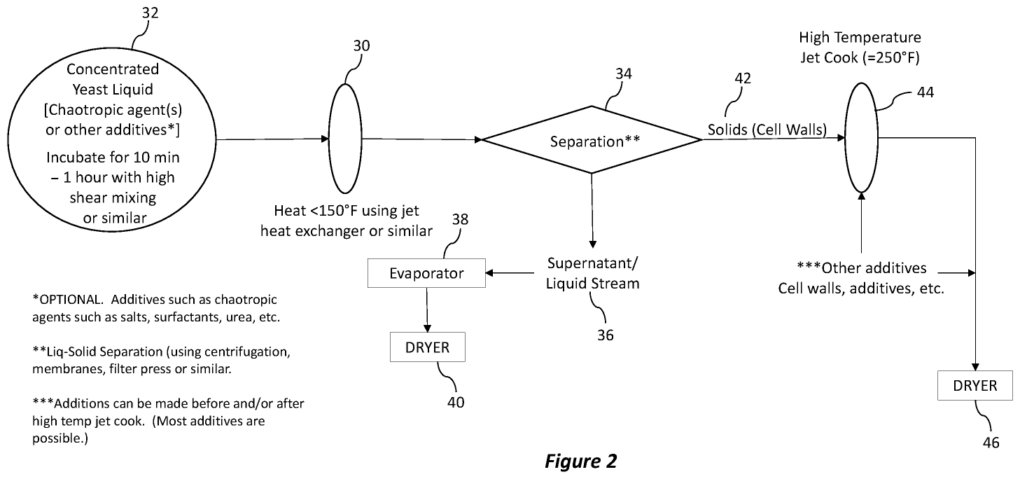

FIG. 2 illustrates a further embodiment where multiple jet cooking stages are used. In this form, a jet cooker is used in a first stage 30 at a temperature below about 150° F. A concentrated yeast liquid 32 is passed to the first stage 30 to cause at least some of the materials to separate from the cells. The material is then separated at reference 34 with separated soluble 36 material passed on to an evaporator 38 and dryer 40. Separated solids 42 can then be sent to a second jet cooking stage 44 that is operated at a temperature of about 250° F. or more. The separated solids 42 can be combined with additives and/or agents prior to and/or after the second jet cooking stage 44. The resulting material from the second jet cooking stage 44 can then be dried in dryer 46.

In some embodiments a chaotropic agent is added to the treatment step to aid, improve, or enhance the rupture and dissociation of the cell wall or otherwise facilitate the recovery and purification of a fraction or biomolecule. The choice of agent will depend on application and product. For instance, urea is a good chaotropic agent for cell lysis. Nonetheless, agents such as urea can only be used in a limited number of feed and niche applications as it can be toxic in non-ruminant animals. Hydrogen peroxide which disrupts cell membranes and walls is more acceptable as it degrades to O₂ and water.

When linked with a separation technology, the process allows for dissociation and fractionation, and recovery of soluble and insoluble fractions. Separation of liquid-solids fractions can be accomplished using various available technologies including but not limited to centrifugation, filtration or related systems. After separation of released constituents, the residual biomass typically containing cell walls, organelles such as starch bodies, chloroplasts and related entities can then be recycled or subjected to one or more disruptive treatments and separation steps.

Dissociation and sequential fractionation are significant as it allows for the dissociation and recovery of the whole cell matter elements without prolonged expose to harsh, degrading conditions which can denature or modifying select categories of compounds. Extremely susceptible cell constituents include most soluble biomolecules, low molecular weight metabolites as well as any active compounds.

In summary, most dissociation processes usually recover a limited number or specific fraction of compounds but are generally unsuccessful at liberating a spectrum of cell constituents resulting in the recovery of a product and many more low value by-products or waste to be disposed. This is not only unsustainable but costly. Aggressive approaches that focus on a finite number of constituents also add significant complexity as they yield a complex heterogeneous mixture. The discovery, recovery and purification of materials from this mixture must then be separated often using multiple analytical methodologies.

As noted above, various products can be obtained from the cells and/or microorganisms in the one or more processing stages described herein. In one form, the process may yield substantially polymeric cytoplasmic proteins and other biomolecules. According to one form, cytoplasmic composition (yeast extract) has a nominal degree of protein and nucleic acid hydrolysis.

### EXAMPLES

### Example 1

### Heat Mediated Sequential Cell Dissociation.

It has previously been demonstrated that jet cooking at high temperatures will dissociate an intact whole cell to yield a diverse mix of soluble and insoluble cell constituents. Fractionation of this mixture is difficult, expensive and suited to laboratory studies as diverse, multiple processes and systems are necessary. In this example, jet cooking at lower temperatures can be used to provide an equilibrium between dissociated components and the intact cell body (or core). Consequently, exposing cells or cell elements such as cell walls to different process temperatures and times enables progressive degrees of cell dissociation. For instance, subjecting an intact cell to two or more different temperature ranges results in controlled, reproducible cell dissociation. When cells are jet cooked at around 160-170° F. for 5-15 minutes the cells are not ruptured but select cell surface materials are released. At 180-200° F. an even greater percentage of solids is liberated; at 210-240° F. cell rupture is evident. When temperatures exceed 250° F. cell rupture is pronounced. Jet cooking at operational temperatures above 305° F. indicate that substantial cell hydrolysis is rapidly achieved. Maintaining cells at high temperatures and pressure as in an autoclave (250° F., 121 psi) is not sufficient to attain the results of the subject invention.

The terminal stage of the subject process is directed at the dissociation of refractory cell elements such as the cell wall, in toto or partially dissociated. By necessity harsher conditions including but not limited to high temperature, high shear, and alkaline or acidic pH treatment are warranted. In particular, high temperature, multi-pass jet cooking is preferred.

Multiple temperature or physical and chemical treatments using a single-stage or a multistage, serial arrangement of temperature steps with recovery of product at each step, as needed, resolves many of the challenges associated with optimizing biomass recovery. An analogous sequential fractionation approach is also relevant to treatment the cell biomass.

Cell dissociation as described herein uses temperature, shear and retention time to efficiently breakup the cell. Unlike autolysis and other enzyme mediated processes though, the cell constituents can be recovered intact.

The process can produce different degrees of dissociation ranging from the disruption of complex intracellular structures and macromolecules such as homo and heteromultimeric proteins, ribosomes, DNA and related assemblages. The disruption of noncovalent bonds associated with the loss of internal structural and molecular integrity results in a random, denatured mixture of biomolecules. The cell wall under these conditions would weaken and leak at the lower temperatures but not break. Retention times of about 5-15 minutes at temperatures ranging from about 160 to 190° F. would cause these changes.

Increasing the temperature to 190 to 230° F. and increasing retention time to 15-30 minutes escalates the loss of structural integrity and also weakening of the foundational wall elements. Under these conditions, leakage of internal cell constituents into the media is increasing.

At higher temperatures such as 240-300° F., the more recalcitrant biomolecules such as interstitial elements, membranes and inner cell wall constituents would collapse and be released into the media as soluble and insoluble entities.

Decomposition of the cell was followed in a stepwise mode at several discrete temperatures. The initial stages of decomposition could be outlined as represented in Table 1. The viscosity and pH are being used as broad indicators cellular events.

TABLE 1

| Samples | Temperature | Viscosity | pH |
|---|---|---|---|
| Control | Not Processed | 13.5 | 3.11 |
| 1 | 180° F. | 70.9 | 3.18 |
| 2 | 200° F. | 70.5 | 3.24 |
| 3 | 220° F. | 50.8 | 3.49 |

As process temperature increased there were concomitant changes in viscosity and pH.

### Example 2

### Physiochemical Enhancements to Thermal Dissociation.

When temperature alone is insufficient to achieve the desired dissociation (or resolution), additives/agents can be incorporated to shift the heat dissociation profile or add greater sensitivity. The inclusion of dissociation agents including but not limited to chaotropic agents, pH, osmotic stressors, surfactants or combinations thereof may facilitate and fine-tune the dissociation process without degrading or modifying targeted constituents such as labile metabolites. In addition to being benign, preferred agents would have some of the following attributes: fast acting, short lived, able to affect broad range of soluble and insoluble biomolecules such as proteins, lipids and carbohydrates. Salts, acids, bases, and such widely used chemicals may be suitable, along with other materials described herein.

The agents could be present throughout entire the dissociation process or added at various stages. For instance, NaCl could be added to the process stream after the low molecular weight, labile metabolites have been recovered in a first stage.

This is significant as it allows for enhanced dissociation at even lower temperatures with subsequent fractionation of the whole cell matter with potentially less denaturing of particular categories of compounds. Labile low molecular weight metabolites as well other active compounds are representative of such a category. Further utility of this approach would have relevance at the industrial (commercial) scale as it reduces energy inputs and cost as well as fine tuning separations.

### Example 3

About 180 gm of dry inactive (dead) Saccharomyces cerevisiae were hydrated by adding to 1 L of water with stirring. The cells were agitated for about 20 minutes. Next, approximately 500 mls of the 17% cell suspension was transferred to a 65° C. water bath with agitation. In about 20 minutes the temperature of yeast solution increased to 65° C. and stabilized.

A sufficient volume of a 35% commercial H₂O₂ was slowly added with mixing to bring the final H₂O₂ concentration to about 9%. The treatment temperature was maintained at 65° C. with mixing for another 30 minutes. The solution was then centrifuged at about 4000 rpm. The supernatant was collected and pooled. The pellets were also collected.

The supernatant samples of the untreated Control and H₂O₂ treated samples exhibited the results shown.

| | HOT WATER | HOT WATER + 9% H₂0₂ |
|---|---|---|
| Supernatant | 1.7% | 7.6% |

The findings show that both the hot water and hot water plus 9% H₂O₂ treatments resulted in release of solids, however, the inclusion of 9% H₂O₂ did expedite the dissociation.

Even at lower concentrations such as 2-3% hydrogen peroxide can induce degradative processes which can effect intracellular structures, membranes as well as cell as cell wall components. The cell morphology may remain unchanged though.

### Example 4

In another embodiment two or more different sources including but not limited to microbial, plant, aquatic, mineral or other soft material sources are co-processed. An exemplary application would be using different fungal strains such as Saccharomyces, Torulopsis, Kluyeromyces, Schizosaccharomyces or bacterial and algal materials, mushroom by-products such as stems and associated edible mass available but not currently used. These and still other sources can be co-processed using the features described herein.

The inventive process and approach is significantly different from a typical blending operation such as 1) mixing separately lysed and dried cells, 2) combining hydrolyzed materials, mixing and drying the entire mixture as well as blending preprocessed cell materials, subjecting to further enzymatic or physiochemical treatment and drying as well as other processing and mixing systems commercially available. While the finished product may be a mix, it is hypothesized the material (or blend) produced by the inventive processes herein will have different functional, chemical, nutritional and other valuable attributes.

By way of another illustration, nutritionists have long recognized that biomasses used in feed and food applications differ. In some instances, the differences can be substantial. Co-processing biomass would dissociate and mix the respective components to resolve inadequacies. Further the co-processed composition may also introduce chemical and functional changes to the finished composition as the components are physically altered without changing the chemical structures.

Further co-processing in the manner found herein could also be substantially less expensive than processing multiple materials that are then blended. Some of these aspects are demonstrated in the following example where different cells are processed concurrently to produce a new composition comprised of biomolecules of the corresponding cell types.

An important distinction between the inventive technology and other methodologies is that cell lysis occurs at temperatures ranging from 180-320° F. in a turbulent, high shear environment. The settings would be variable and process dependent. Hence range of new bioactives could be generated. Further it would be expected that the cell structures would collapse independent of the source to some extent.

Hence because of the distinctive reaction environment and bioenergetic conditions the potential to produce unique functionalities and compositions are available. It is further proposed that a thermally activated, denaturing chemical environment may also be capable of producing synergies at the molecular level unlike other blending technologies or systems.

Co-processing of yeast cell walls and fermented bacterial biomass at a 50:50 ratio was successfully demonstrated using Schizosaccharomyces cell walls and a laboratory jet cook system. The following parameters were used:

Liquid cell walls were mixed with bacterial cell biomass for 20-30 minutes using an overhead mixer. The final mixture consisted of about 50% fungal and 50% bacterial (dry solids basis). The blend was transferred to the cooker blend tank and mixed for another few minutes at room temperature. The temperature, pressure, flow rate and retention conditions in the system were adjusted using water. Once stabilized the valve to holding the holding tank was opened and sample pumped into the system. While filling the equipment was monitored and fine-tuned equipment. After the water had been displaced, as indicated by seeing sample at the exit, sample collection was initiated. The first few hundred milliliters collected were discarded; thereafter sample collection was initiated. Samples were collected in 0.5-1 L containers and set aside to cool.

The processed liquid composition appeared homogeneous with no clumping or precipitation. Further the viscosity of the treated composition was higher than the untreated liquid suggesting that the yeast cell wall had been disrupted.

The liquid sample was next freeze-dried in a laboratory dryer. The dried powder was analyzed for % protein as well as the glucan and mannanoligosaccharide (MOS) content. The following results were obtained:

Protein: 55%

Glucan: 6.9%

MOS: 7.6%

The protein content was higher than the anticipated value, ˜47% which was the average of the two materials used in the original liquid. The glucan and MOS were lower than anticipated.

The inventive technology and process can produce a single composition:

with multiple attributes and functionalities,

improve nutritional as well as organoleptic properties,

produce and enhance synergies of individual components beyond the practices and scope of conventional mixing and blending systems.

### Example 5

### Co-Processed Yeast: Poultry Feeding Study

Saccharomyces cerevisiae dried cell wall powder and whole cell Torula (Candida utilis) yeast powder were used to make a 50:50 liquid mix. The composition was blended for about 30 minutes using an overhead mixer. The solids content of the finished liquid was approximately 13%.

The blended yeast liquid was processed on a pilot scale jet cooker. The high temperature physicochemical processing used the following conditions:

Temperature: approx. 310° F.

Pressure: 60-70 psig

Retention Time: 15-20 minutes

The processed liquid was collected in buckets, remixed and recycled under the same conditions. After the second pass the liquid was again pooled and mixed. Samples recovered demonstrated that the finished solids content was approximately 10.5%.

Spray drying of the co-processed yeast was attempted but abandoned due to equipment issues. In the absence of drying, the product was cooled to room temperature and then transferred to a cold room. The product was kept in the cold room until the poultry feeding trials.

Feeding Trial

Earlier studies had shown hydrolyzed Saccharomyces cerevisiae dried cell wall powder exhibited small improvements in weight gain, feed efficiency and other feed associated metrics. Similarly, the addition of hydrolyzed Torula powder to chick diets also demonstrated a positive growth response.

The co-processed Saccharomyces and Torula hydrolysate when added to chick diets at the same dry solids levels also exhibited similar positive responses.

Bacterial 165 rRNA Microbiome Analysis

To investigate the effects of each of the three treatments and control on the gut microbiome an analysis of microbial DNA based sequence data was used, specifically 16S rRNA Microbiome Analysis. The methodology is widely used to identify and catalog the microbial diversity, relative abundance of each bacterial genus as well as changes in the gut microbiome of animals. This methodology is also used to investigate cause and effect interactions. For example, the effect of a food, drug or other associated reaction on essential microbial populations.

Poultry ceca was used to investigate effects of the four treatments. Poultry ceca samples from the three aforementioned feeding trials and control samples were collected after the trial and immediately frozen. The four frozen samples were then submitted for microbiome analysis.

The chicken microbiota study used the ceca as the sampling location due to the specific role of the ceca microbiota in chicken productivity, health and wellbeing. However, sampling from the ceca required that a set number of birds from each feeding trial be sacrificed. The ceca of four birds were recovered and the content pooled. The collected contents were immediately frozen and stored at approximately −30° F. The samples remained frozen until being shipped to the bio diagnostics facility.

This testing was conducted to determine the effects of the four feed treatments on the poultry intestinal tract. The findings showed that the treatments did not impact 1) the number of different bacterial species found in the extracted cecum samples or the evenness of species abundance present; 2) there was no major impact on the overall community structure of the intestinal tract.

However, some treatments effects were observed on specific bacterial genera. Specifically, the relative abundance of Faecalibacterium prausnitzii was higher in the cecum of birds receiving the co-processed Saccharomyces cerevisiae dried cell wall—whole cell Torula (Candida utilis) blend than the other treatments. Other changes were also observed that were associated with specific treatments. Most of these were not significant and did rise to the level of substantive treatment induced effects.

Faecalibacterium prausnitzii plays a major role in gut health as documented in the scientific and patent literature. Sokol, H. et al. (2008). Faecalibacterium prausnitzii is an anti-inflammatory commensal bacterium identified by gut microbiota analysis of Crohn disease patients. Proc. Natl. Acad. Sci. U.S.A. 105, 16731-16736. doi: 10.1073/pnas.0804812105. Characterization of Novel Faecalibacterium prausnitzii Strains Isolated from Healthy Volunteers: A Step Forward in the Use of Faecalibacterium prausnitzii as a Next Generation Probiotic. Martin et al. Front. Micrbiol. 8:1226. See also U.S. Pat. Nos. 10,960,033 and 10,918,678.

The inventive co-processing methodology offers an unorthodox approach to use new substrates and nutrient compositions to alter the gut microbiota.

The methods and systems described herein can be performed in any suitable order unless otherwise indicated herein. The use of any and all examples, or language describing an example (e.g., “such as”) provided herein, is intended to illuminate the invention and does not pose a limitation on the scope of the invention. Any statement herein as to the nature or benefits of the invention or of the preferred embodiments is not intended to be limiting. This invention includes all modifications and equivalents of the subject matter recited herein as permitted by applicable law. Moreover, any combination of the above-described elements in all possible variations thereof is encompassed by the invention unless otherwise indicated herein or otherwise clearly contradicted by context. The description herein of any reference or patent, even if identified as “prior,” is not intended to constitute a concession that such reference or patent is available as prior art against the present invention. No unclaimed language should be deemed to limit the invention in scope. Any statements or suggestions herein that certain features constitute a component of the claimed invention are not intended to be limiting unless reflected in the appended claims. Neither the marking of the patent number on any product nor the identification of the patent number in connection with any service should be deemed a representation that all embodiments described herein are incorporated into such product or service.

What is claimed is:

1. A method for extraction of cell components from a plurality of cells comprising:
subjecting the plurality of cells to heat, pH, and shear in a first step under conditions to separate one or more cellular components from the cells, the temperature being less than about 170° F.

2. The method of claim 1 wherein the plurality of cells is cooked in a jet cooker.

3. The method of claim 1 wherein the plurality of cells is subjected to a second processing step wherein the temperature is greater than 170° F.

4. The method of claim 3 wherein the second processing step includes cooking in a jet cooker.

5. The method of claim 3 wherein at least a portion of the one or more cellular components are separated from a remainder of the cellular components with the remainder of the cellular components being subjected to the second processing step.

6. The method of claim 1 further comprising combining the plurality of cells with a chaotropic agent.

7. The method of claim 1 wherein the plurality of cells is subjected to a temperature of less than about 170° F. for less than about 15 minutes.

8. The method of claim 1 wherein polysaccharides are recovered from the plurality of cells.

9. The method of claim 1 wherein the plurality of cells includes a plurality of different types of cells.

10. The method of claim 1 wherein the temperature is less than about 150° F.

11. The method of claim 1 wherein the plurality of cells is subjected to a pressure of less than about 50 psi.

12. The method of claim 1 wherein the plurality of cells include cell walls and the first step does not substantially rupture the cell walls.

13. A method for extraction of cell components from a plurality of different types of cells comprising:
providing a combination of a first type of cells and a second type of cells to form a combination of the plurality of different types of cells; and
subjecting the combination of the plurality of different types of cells to heat, pH, and shear in a first step under conditions to separate one or more cellular components from the cells.

14. The method of claim 13 further comprising subjected the plurality of different types of cells to heat, pH, and shear in a second step, the temperature in the first step being less than about 170° F. and the temperature in the second step being greater than 170° F.

15. The method of claim 14 wherein at least one of the first and second steps includes cooking in a jet cooker.

16. The method of claim 13 further comprising adding a chaotropic agent to the combination of the plurality of different types of cells.

17. The method of claim 13 wherein the temperature in the first step is less than about 150° F.

18. The method of claim 13 wherein the plurality of cells is subjected to a pressure of less than about 50 psi in the first step.

19. The method of claim 13 wherein the plurality of different types of cells include cell walls and the first step does not substantially rupture the cell walls.

20. A method of providing nutrition to an animal comprising the steps of:
providing a nutritional composition to an animal, the nutritional composition having been prepared by a process of subjecting a plurality of cells to heat, pH, and shear in a first step under conditions to separate one or more cellular components from the cells, the temperature being less than about 170° F.
